(19) United States
(12) Patent Application Publication
(10) Pub. No.: US 2014/0286890 A1
(43) Pub. Date: Sep. 25, 2014

(54) COMPOSITIONS AND METHODS FOR ERADICATION OF ODORS

(71) Applicant: NBIP, LLC, Plano, TX (US)

(72) Inventors: Burt R. Sookram, Palm Harbor, FL (US); John W. Veenstra, Plano, TX (US)

(73) Assignee: NBIP, LLC, Plano, TX (US)

(21) Appl. No.: 14/356,597

(22) PCT Filed: Nov. 6, 2012
(86) PCT No.: PCT/US2012/063673

§ 371 Date: May 6, 2014

Related U.S. Application Data
(60) Provisional application No. 61/556,255, filed on Nov. 6, 2011.

(51) Int. Cl. A61L 9/01

(52) U.S. Cl. CPC A61L 9/01
USPC 424/76.8

(57) ABSTRACT

Compositions and methods for controlling odors associated with animals and an animal's excrement, and environmental odors, comprising a biocidal system comprised of a primary biocide, a pH buffer agent, a surfactant, all in an aqueous based carrier.

### CROSS-REFERENCES TO RELATED APPLICATIONS

This Application claims the benefit under 35 U.S.C. §119(e) of U.S. Provisional Patent Application Ser. No. 61/556,255, filed Nov. 6, 2011, which is incorporated herein by reference in its entirety as if fully set forth herein.

### FIELD OF THE INVENTION

The present disclosure relates to compositions and methods that are effective in controlling and preventing odors.

### BACKGROUND OF THE INVENTION

There are several sources of odors that can occur including odors arising from food preparation, smoke and animal odors such as urine, feces, breath, fur, feathers and the sorts. The most common problems of animal odors originate from urination and feces. Thus, it would be desirable to have a composition that is capable of eradicating odors from any source, particularly animal and animal excrement odors.

### SUMMARY OF THE INVENTION

The claimed compositions and methods provide a method for preventing and controlling odors including animal odors. Compositions of the invention prevent and control ammonia build-up while removing the bacteria causing the odor. These odors may be associated with animals and the odors that come from urine and feces from animals. The compositions and methods are suitable for use with any animal including, but not limited to pets, domesticated and non-domesticated animals and humans. Composition and methods are also suitable for use with odors associated with food preparation, cooking, smoke and other airborne odors.

The disclosed compositions comprise a biocidal system comprising a primary biocide, a pH buffer and a surfactant, present in an aqueous composition.

### DETAILED DESCRIPTION OF EXEMPLARY EMBODIMENTS

In this specification and in the claims that follow, reference will be made to a number of terms that shall be defined to have the following meanings:

The term “animal” as used herein means all pets, domesticated and non-domesticated animals, humans and all living beings that produce body odors or body fluids or excrement.

The term “excrement” as used herein means the urine or feces emanating from an animal.

Throughout this specification, unless the context requires otherwise, the word “comprise,” or variations such as “comprises” or “comprising,” will be understood to imply the inclusion of a stated integer or step or group of integers or steps but not the exclusion of any other integer or step or group of integers or steps.

It must be noted that, as used in the specification and the appended claims, the singular forms “a,” “an” and “the” include plural referents unless the context clearly dictates otherwise. Thus, for example, reference to “a carrier” includes mixtures of two or more such carriers, and the like.

The term “effective amount” as used herein means “an amount of a composition as disclosed herein, effective at dosages and for periods of time necessary to achieve the desired results.

“Optional” or “optionally” means that the subsequently described event or circumstance can or cannot occur, and that the description includes instances where the event or circumstance occurs and instances where it does not.

Ranges may be expressed herein as from “about” one particular value, and/or to “about” another particular value. When such a range is expressed, another aspect includes from the one particular value and/or to the other particular value. Similarly, when values are expressed as approximations by use of the antecedent “about,” it will be understood that the particular value forms another aspect. It will be further understood that the endpoints of each of the ranges are significant both in relation to the other endpoint and independently of the other endpoint.

A “weight percent” of a component, unless specifically stated to the contrary, is based on the total weight of the formulation or composition in which the component is included.

“Contacting” as used herein refers to applying a composition to an animal or an animal's excrement.

“Sufficient amount” and “sufficient time” as used herein refers to an amount and time needed to achieve the desired result or results, such as control and/or prevention of odors of an animal.

“Admixture” or “blend” as generally used herein means a physical combination of two or more different components.

“Controlled release” as used herein means the use of a material to regulate the release of another substance.

“Excipient” is used herein to include any other compound that may be contained in or on the microparticle, which is not a therapeutically or biologically active compound. As such, an excipient should be pharmaceutically or biologically acceptable or relevant (for example, an excipient should generally be non-toxic to the subject). “Excipient” includes a single such compound and is intended to include a plurality of excipients.

“Odor” is used herein to refer to any pungent smell or stench including bacterial and ammonia odors, associated with animals and their excrement, and other odors, such as environmental odors, including food preparations, cooking and smoke.

“Primary biocide” is used herein to refer to compounds that are biologically active against a primary pathogen.

“Primary pathogen” is used herein to refer to bacteria, viruses fungi or other biologically active microorganisms.

The present disclosure addresses solutions to several unmet needs as defined below:

1. Providing compositions effective in killing one or more bacterial pathogens. Non-limiting examples include enterococci, group B. streptococci, E faecalis, coagulase negative staphylococci, Escherichia coli, (E. coli) and Klebsiella pneumoniae.

2. Providing compositions effective against environmental bacterial pathogens. Non-limiting examples include Streptococcus spp, Escherichia coli, Klebsiella species, A. pyogenes and Pseudomonas species.

3. Providing compositions effective against bacterial caused odors such as hydrogen sulfide, methyl mercaptan, allylmethyl sulfide and dimethyl sulfide.

4. Providing compositions effective in spread of one or more odor-causing bacterial pathogens from an infected animal to other animals or an apparatus that contacts any infected animals.

5. Reducing the ammonia from an animal's excrements.

6. Reducing and eliminating ammonia odor, food preparation odor, cooking odor and smoke odor.

7. Reducing and eliminating other animal odors such as breath, fur and feathers.

Treatment Compositions

An embodiment of the invention is directed to a composition for reducing odors associated with excrement, breath , fur and feather where ammonia and other mal-odor build up is observed in animals comprising:

a. from about 0.01% to about 20.0% by weight of a biocidal system comprising:
i. from about 0.01% to about 25% by weight of a primary biocide; and
ii. at least about 0.01% to about 25% by weight of a pH buffer, where the pH buffer is a biocidal, dermal, non-corrosive acid composition, having a proton count range of from 1.0×10²⁰ to 9.9×10²⁶, an embodied conductivity range of from 250 mV to 1500 mV and has a pH of less than 2.0 when the pH buffer is present at a concentration of 0.1% by weight.; and

b. from about 0.01% to 2% by weight of a surfactant; and

c. the balance being an aqueous based carrier.

However, other non-limiting embodiments and combinations are possible as further disclosed herein.

Biocidal System

The disclosed compositions comprise a biocidal system. The biocidal system comprises a primary biocide and a pH buffer.

Primary Biocide

A first group of suitable biocides include quaternary ammonium compounds chosen from (C₁₂-C₁₄ alkyl)(C₁-C₂ dialkyl)benzyl ammonium salts, N-(C₁₂-C₁₈ alkyl)heteroaryl ammonium salts, and N-[(C₁₂-C₁₄ alkyl)(C₁-C₂ dialkyl)]heteroarylalkylene ammonium salts. Non-limiting examples of the (C₁₂-C₁₄ alkyl)(C₁-C₂ dialkyl)benzyl ammonium salts include (C₁₂-C₁₄ alkyl)dimethyl-benzyl ammonium chloride, (C₁₂-C₁₄ alkyl)dimethylbenzyl ammonium bromide, and (C₁₂-C₁₄ alkyl)dimethylbenzyl ammonium hydrogen sulfate. Non-limiting examples of the N-(C₁₂-C₁₈ alkyl)heteroaryl ammonium salts include cetyl pyridinium chloride, cetyl pyridinium bromide, and cetyl pyridinium hydrogen sulfide. For the N-(C₁₂-C₁₈ alkyl)heteroaryl ammonium salts other anions can be used.

Further examples of quaternary ammonium compounds suitable for use as the primary biocides include cetyltrimethylammonium chloride, stearyltrimethylammonium chloride, isostearyltrimethylammonium chloride, lauryltrimethylammonium chloride, behenyltrimethyl-ammonium chloride, octadecyltrimethylammonium chloride, cocoyltriinethylammonium chloride, cetyltrimethylammonium bromide, stearyltrimethylammonium bromide, lauryltrimethylammonium bromide, isostearyllauryldimethylammonium chloride, dicetyldimethylammonium chloride, distearyldimethylammonium chloride, dicocoyldimethylammonium chloride, gluconamidopropyldimethylhydroxyethylammonium chloride, di[polyoxyethylene(2)]oleylmethylammonium chloride, dodecyldimethylethylammonium chloride, octyldihydroxyethylmethylammonium chloride, tri[polyoxyethylene(5)]-stearylammonium chloride, polyoxypropylenemethyldiethylammonium chloride, lauryldimethyl(ethylbenzyl)ammonium chloride, behenamidopropyl-N,N-dimethyl-N-(2,3-dihydroxypropyl)ammonium chloride, tallowdimethylammoniopropyltrimethylammonium dichloride, and benzalconium chloride.

A second group of suitable biocides includes copper, zinc, and silver, salts of chlorides, chlorites, perchlorates, hypochlorites, sulfates, sulfites, bisulfates, and bisulfites. Also, colloid metal such as silver, gold, copper and zinc have superior biocidal properties. Colloidal silver, gold, copper, and zinc are extracted and created as ultrafine (0.010-0.001 micron) particles.

A third group of suitable biocides includes organic acids which are safe under the FDA GRAS guidelines for food production yet still effective in controlling bacteria.

The first group of suitable organic acids is Lactic, Acetic, Formic, Fumaric, Citric, Oxalic, Adipic, and Uric.

The second group of suitable organic acids is the carboxylic acids, whose acidity is associated with their carboxyl group —COOH. Sulfonic acids, containing the group —SO₂OH, are relatively stronger acids. The relative stability of the conjugate base of the acid determines its acidity. In some biological systems more complex organic acids such as L-lactic, citric, and D-glucuronic acids are formed. These use the hydroxyl or carboxyl group.

The third group of suitable organic acids are Humic, Sebacic, Stearic, Gallic, Palmitic, Caffeic, Glyoxylic, Fulvic, Carnosic, Anthranilic, Ellagic, Lipoic, Chlorogenic, Rosmarinic, Phosphoric, Methacrylic, Oleanic, Nitrohumic, Florocinnamic, Hexaflorosilicic, Hydrofluoric, Hydroxycitric and Silicofluoric.

The fourth group of suitable organic acids is fruit acids. The acids in fruits are chiefly acetic, malic, citric, tartaric, oxalic, and in some instances boric. Malic acid is present in apples, pears, currants, blackberries, raspberries, quince, pineapple, cherries, and rhubarb. Citric acid is found in lemons, oranges, grapefruit, lemons, limes, quince, gooseberry, strawberry, raspberry, currant, and cranberry. Tartaric acid occurs in grapes. Boric acid is found in many fresh fruits and vegetables. Mandelic acid is present in almonds.

The fifth group of suitable organic acids is beta hydroxy acids, which is a type of phenolic acid. Salicylic acid is a colorless crystalline organic acid whose main active ingredient obtained from this source is a monohydroxiybenzoic acid.

The sixth group of suitable organic acids is a class of products that break biofilm. Biofilms are the protective layer/barrier that surround bacteria. Some species are not able to attach to a surface on their own but are often able to anchor themselves to the matrix or the bacteria cells. It is during this colonization that the cells are able to communicate via its quorum sensing ability. Once colonization has begun, the biofilm grows through a combination of cell division and recruitment. The final stage of biofilm formation is known as development and is the stage in which the biofilm is established and may only change in shape and size. The development of a biofilm may allow an aggregate cell colony to be increasingly resistant. A biofilm's hard protective surface can be broken by Lactobacillus sc Nisin which is produced by fermentation using the bacterium Lactococcus lactis. This is obtained from the culturing of Lactococcus lactis on natural substrates, such as milk or dextrose, and is not chemically synthesized. This is a peptide which is produced by the food grade dairy starter bacterium Lactococcus lactis.

A seventh group of suitable organic acids is natural enzymes. Enzymes are proteins that catalyze chemical reactions and range from just 62 amino acid residues. Typically, these are protease, lipase, diastase, and cellulase enzymes. Enzymes are usually very specific as to which reactions they catalyze and the substrates that are involved in these reactions. The shape, charge, and hydrophilic/hydrophobic nature characterize the enzymes.

pH Buffer

The pH buffer is a biocidal, dermal, non-corrosive acid composition, having a proton count range of from 1.0×10²⁰ to 9.9×10²⁶, an embodied conductivity range of from 250 mV to 1500 mV and a 0.1% solution of the composition having a pH of under 2.0.

Surfactant

A surfactant component includes ingredients that modify the water in the system making it suitable from use with several types of water such as hard water, soft water, sulfite contaminated water, rain water, pond water, well water or calcium rich water. This allows the embodiment composition to cling or stick to the animal's hair or skin, or excrement. Cationic surfactants of the quaternary ammonium compound referred to as “poly quats” are used in many hair shampoos and conditioners. These agents improve the body of the hair and reduce its static charge. Surfactant products often used for human hair treatment provide the cling or stickiness necessary for the application of the biocidal compositions to animals. Quaternary ammonium compounds which are more compatible with anionic surfactants generally have an inadequate conditioning effect.

In-order to improve the ability for an aqueous mixture to adhere to an animal, or its excrement, dialkyl diallyl ammonium chloride/acrylic acid-type polymers is added. This method for improving the stickiness properties of the composition encompasses adding an effective amount of a polymer comprising:

1. About 60% to about 99%, based on total polymer weight, of a quaternary diallyl dialkyl ammonium monomer, where alkyl groups are independently selected from alkyl groups of 1 to 18 carbon atoms, preferably C₁₋₄ alkyl, and where said quaternary diallyl dialkyl ammonium monomer's counterion is selected from the group consisting of conjugate bases of acids having an ionization constant greater than 10⁻¹³ , more preferably selected from the group consisting of fluoride, chloride, bromide, hydroxide, nitrate, acetate, hydrogen sulfate, and primary phosphates; and 2. About 1% to about 40%, based on total polymer weight, of an anionic monomer selected from the group consisting of acrylic acid and methacrylic acid; where the average molecular weight of said polymer ranges from about 50,000 to about 10,000,000, as determined by gel permeation chromatography.

The polymer base can also be a combination of one or more bases, for example, glycerol in combination with ethoxylated partial glyceride fatty acid esters. These include branched chain esters, ethoxylated partial glyceride fatty acid esters, protein derivatives, lanolin and lanolin derivatives, and fatty alcohol ethoxylates, emollient oils, fatty acids, fatty alcohols, and their esters. Other examples of suitable bases include glycerin, sortibal aloe, poylglycols, polyethylene glycol, polyoxyethylene and polyethylene oxide.

Carriers

The balance of the disclosed compositions comprises a carrier. The carrier can be any suitable material that can dissolve the active ingredients and co-ingredients and deliver the biocidal system to the infected areas of the animal or animals excrement being treated. Water is a convenient carrier for liquid embodiments of the disclosed composition.

Formulations

The following are non-limiting examples of the disclosed compositions:

TABLE 1

| Ingredients | Amount (wt %) |
|---|---|
| Malic Acid | 0.10-0.75 |
| pH buffer | 0.05-1.00 |
| Thickener | 0.25-4.00 |
| color | trace |
| carrier | balance |

In certain embodiments of the invention, the thickener present in the composition set forth in Table 1 is replaced with a foaming agent (1.25 wt %).

TABLE 2

| Ingredients | Amount (wt %) |
|---|---|
| Cetyl pyridinium chloride | 0.10-1.00 |
| pH buffer | 0.05-1.00 |
| PEG 6 | 0.35-5.00 |
| Thickener | 0.25-4.00 |
| color | trace |
| carrier | balance |

Methods of Use

The disclosed compositions can be used in various environments of an animal. One example would be a cat litter box. Application methods and dosage regimens are dictated by the frequency of urine and/or feces deposits. The reason it is based on the frequency of the urination is the litter boxes size and the amount of litter materials stored in the boxes are generally accessible to the pets. Litter boxes must be deep enough so when an application is done the pet usually has enough materials at least about 1 to 3 inches of composition or enough to cover any materials expelled by the pet. A second example would be a poultry pen which accumulates chicken excrement over the life of the chicken. In these two examples and all other examples, the biocidal composition must be evenly sprayed, foamed, or fogged to make contact with any urine or feces deposited by the animal.

1. Spray applications are effective where the liquid composition can be applied directly to the litter material containing in the animal's excrement.

2. Foam applications can be utilized as a replacement for the liquid spray composition. The main advantage of the foam is its thickness, which will allow the embodied composition a longer contact period with the litter material, allowing longer penetration.

3. A fog application can be used to apply the embodied composition in a manner that will allow the excrement to be totally engulfed thereby allowing the biocidal composition the longest contact period.

The disclosed compositions can also be used to reduce odor originating from breath, fur and feathers of an animal. The biocidal composition must be evenly sprayed, foamed, or fogged to make contact with any source of the odor.

The disclosed compositions can be used to reduce the odor from food preparations, cooking and/or smoke. The preferred application for food preparations, cooking and/or smoke is either a spray or fog depending upon the severity of the odor problem.

Working Examples

The following procedures can be used to evaluate the disclosed compositions against various microorganisms. The composition tested in each of the below examples comprised 0.25 wt % malic acid, 0.10 wt % pH buffer, and 0.5 wt % thickener. The results below further indicate the effectiveness of the disclosed compositions.

Bacterial testing was completed at Biological Consulting Services of North Florida, Inc. on E. coli, and E. faecalis. The results are listed in Table A.

TABLE A

| Sample | Control units (cfu/ml) | Results units (cfu/ml) |
|---|---|---|
| E. coli | 9.3 × 10⁵ | <0.5 |
| Enterococcus faecalis | 1.1 × 10⁶ | <0.5 |

A Draegar Pump and ammonia detection Draegar tubes were used to determine the gas levels. The Draegar pump is a standard bellow style gas sampling pump. It drags the gas a calibrated 100 ml sample through a short-term Draegar tubes. Table B shows the results of ammonia expressed as parts per million (ppm). Urine samples were measured for ammonia gas before and after a spray, application was done on cat urine. The study times were less than 1 hour and 5 days. The table below shows those results:

TABLE B

| | | |
|---|---|---|
| Urine | <1 Hour | 5 Days |

| Samples | Before | After | Before | After |
|---|---|---|---|---|
| Sample 1 | 15 ppm | 2 ppm | 170 ppm | 4 ppm |
| Sample 2 | 18 ppm | 2 ppm | 210 ppm | 5 ppm |
| Sample 3 | 15 ppm | 2 ppm | 200 ppm | 4 ppm |
| Sample 4 | 20 ppm | 2 ppm | 190 ppm | 4 ppm |
| Sample 5 | 20 ppm | 2 ppm | 210 ppm | 5 ppm |

Thus, it can be seen that compositions of the claimed invention are capable of reducing the presence of ammonia gas by about 90% to 98% depending upon the time of treatment, i.e., less than one (1) hour or five (5) days. Treatment times of as little as ten (10) minutes can have results at or near the results of those measured at one (1) hour.

While particular embodiments of the present disclosure have been illustrated and described, it would be obvious to those skilled in the art that various other changes and modifications can be made without departing from the spirit and scope of the disclosure. It is therefore intended to cover in the appended claims all such changes and modifications that are with the scope of this disclosure.

What is claimed is:

1. A composition for reducing odors comprising:
a. from about 0.01% to about 20.0% by weight of a biocidal system comprising:
i. from about 0.01% to about 25% by weight of a primary biocide; and
ii. at least about 0.01% to about 25% by weight of a pH buffer, where the pH buffer is a biocidal, dermal, non-corrosive acid composition, having a proton count range of from 1.0×10²⁰ to 9.9×10²⁶, an embodied conductivity range of from 250 mV to 1500 mV and has a pH of less than 2.0 when the pH buffer is present at a concentration of 0.1% by weight; and
b. from about 0.01% to 2% by weight of a surfactant; and
c. the balance being an aqueous based carrier.

2. The composition according to claim 1, where the primary biocide is a quaternary ammonium salt comprising at least one aryl or heteroaryl unit.

3. The composition according to claim 1, where the primary biocide is chosen from (C ₁₂-C₁₄ alkyl)(C₁-C₂ dialkyl)benzyl ammonium salts, N-(C₁₂-C₁₈ alkyl)heteroaryl ammonium salts, and N-[(C₁₂-C₁₄ alkyl)(C₁-C₂ dialkyl)]heteroarylalkylene ammonium salts.

4. The composition according to claim 1, where the primary biocide is chosen from (C₁₂-C₁₄ alkyl)dimethylbenzyl ammonium chloride, (C₁₂-C₁₄ alkyl)dimethylbenzyl ammonium bromide, (C₁₂-C₁₄ alkyl)dimethylbenzyl ammonium hydrogen sulfate, cetyl pyridinium bromide, and cetyl pyridinium hydrogen sulfide.

5. The composition according to claim 1, where the primary biocide is cetyl pyridinium chloride.

6. The composition according to claim 1, where the primary biocide is chosen from copper, zinc, silver, salts of chlorides, chlorites, perchlorates, hypochlorites, sulfates, sulfites, nitrates, nitrites and hydroxides.

7. The composition according to claim 1, wherein the primary biocide is chosen from fruit acids such as acetic, malic, citric, tartaric, oxalic, tartaric, mandelic and boric.

8. The composition according to claim 1, wherein the primary biocide is chosen from organic acids such as Lactic, Acetic, Formic, Fumaric, Adipic, Citric, Oxalic, or Uric.

9. The composition according to claim 1, wherein the primary biocide is chosen from several organic acids containing carboxylic and/or Sulfonic acids such as Humic, Sebacic, Stearic, Gallic, Palmitic, Caffeic, Glyoxylic, Fulvic, Carnosic, Anthranilic, Ellagic, Oleanic, Lipoic, Chlorogenic, Rosmarinic, Phosphoric, Methacrylic, Nitrohumic, Florocinnamic, Hexaflorosilicic, Hydrofluoric, Hydroxycitric and Silicofluoric.

10. The composition according to claim 1, wherein the primary biocide is chosen from suitable organic acids that are beta hydroxy acids such as Salicylic acid.

11. The composition according to claim 1, wherein the primary biocide is chosen from several natural and/or organic acids that break the biofilm such as acids from fermented Lactococcus lactis, Lactococcus cremoris, Lactococcus hordniae, Lactococcus garvieae, Lactococcus platarum, Lactococcus raffinolactis, or Lactococcus piscium products.

12. The composition according to claim 1, wherein the primary biocide is chosen from a wide range of natural enzymes such as proteolytic, amylolytic, cellulase, papin, invertase, lipolytic, pepsin, bromelain and lactase.

13. The composition according to claim 1, where the surfactant is chosen from about 60% to about 99%, based on total polymer weight, of a quaternary diallyl dialkyl ammonium monomer, wherein alkyl groups are independently selected from alkyl groups of 1 to 18 carbon atoms, C₁₋₄ alkyl, and wherein said quaternary diallyl dialkyl ammonium monomer's counterion is selected from the group consisting of conjugate bases of acids having an ionization constant greater than 10⁻¹³ , selected from the group consisting of fluoride, chloride, bromide, hydroxide, nitrate, acetate, hydrogen sulfate and primary phosphates.

14. The composition according to claim 1, where the surfactant is chosen from about 1% to about 40%, based on total polymer weight, of an anionic monomer selected from the group consisting of acrylic acid and methacrylic acid, wherein the average molecular weight of said polymer ranges from about 50,000 to about 10,000,000, as determined by gel permeation chromatography.

15. The composition according to claim 1, where the surfactant is chosen from a combination of one or more polymer bases, such as glycerol, glycerine, sortibal aloe, polyglycols, polyethylene glycol, polyoxyethylene and polyethylene oxide, in combination with ethoxylated partial glyceride fatty acid esters such branched chain esters, ethoxylated partial glyceride fatty acid esters, protein derivatives, lanolin and lanolin derivatives, and fatty alcohol ethoxylates, emollient oils, fatty acids, fatty alcohols and their esters.

16. The composition according to claim 1, where the cationic or ionic surfactant has an HLB of from about 12 to about 18.

17. The composition according to claim 1, where the cationic or ionic surfactant has an HLB of from about 13 to about 16.
